(19) United States
(12) Patent Application Publication
(10) Pub. No.: US 2003/0162293 A1
(43) Pub. Date: Aug. 28, 2003

(54) Cell transfection compositions comprising genetic material, an amphipathic compound and an enzyme inhibitor and method of use

(76) Inventors: Yong Liang Chu, Rockville, MD (US); Wan-Ching Lai, Rockville, MD (US); Jian-Tai Qiu, Rockville, MD (US); Frank Q. Li, Montgomery Village, MD (US)

Correspondence Address: Supervisor, Patent Prosecution Services, PIPER RUDNICK LLP, 1200 Nineteenth Street, N.W., Washington, DC, 20036-2412, US

(21) Appl. No.: 10/163,300

(22) Filed: Jun. 7, 2002

Related U.S. Application Data
(63) Continuation-in-part of application No. 10/035,223, filed on Jan. 4, 2002.

(51) Int. Cl. C12N 15/88; C07J 41/00

(52) U.S. Cl. 435/458; 554/51; 552/531; 514/44

(57) ABSTRACT

Cell transfection compositions including an amphipathic compound and an enzyme inhibitor such as a histone deacetylase inhibitor for delivery of genetic material into cells are provided. The cell transfection compositions can express high levels of an encoding protein with minium cytotoxicity. Exemplary histone deacetylase inhibitors include trichostatin A (TSA), FR901464, FR901228, trapoxin A (TPX). The amphipathic compounds can be cationic compounds, neutral compounds or combinations thereof. The enzyme inhibitor can be encapsulated in a liposome formed by the amphipathic compound or the enzyme inhibitor can be mixed with a pre-formed liposome of the amphipathic compound.

This application is a Continuation-In-Part of U.S. patent application Ser. No. 10/035,223, filed Jan. 4, 2002, the entirety of which is incorporated herein by reference.

### BACKGROUND OF THE INVENTION

1. Field of the Invention

This invention relates generally to transfection compositions and methods of use. More specifically, the present invention relates to compositions comprising amphipathic compounds and enzyme inhibitors (e.g., histone deacetylase inhibitors) and to methods of using these compositions for delivery of genetic material (e.g., polynucleotides) into cells.

2. Background of the Technology

Various methodologies have been used to transfect macromolecules such as DNA into cells. These methods include microinjection, protoplast fusion, liposome fusion, calcium phosphate precipitation, electroporation and retroviruses. All of these methods suffer from significant drawbacks: they tend to be too inefficient, too toxic, too complicated or too tedious to be conveniently and effectively adapted to biological and/or therapeutic protocols on a large scale. For instance, the calcium phosphate precipitation method can successfully transfect only about 1 in 10⁷ to 1 in 10⁴ cells. This frequency is too low to be applied to current biological and/or therapeutic protocols. Microinjection is efficient but not practical for large numbers of cells or for large numbers of patients. Protoplast fusion is more efficient than the calcium phosphate method but the polyethylene glycol that is required is toxic to the cells. Electroporation is more efficient than calcium phosphate but requires a special apparatus. Retroviruses are sufficiently efficient but the introduction of viruses into the patient leads to concerns about infection and cancer.

Lipid aggregates (e.g., liposomes) have also been found to be useful as agents for delivery to introduce macromolecules, such as DNA, RNA, protein, and small chemical compounds such as pharmaceuticals, into cells. In particular, lipid aggregates comprising cationic lipid components have been shown to be especially effective for delivering anionic molecules into cells. In part, the effectiveness of cationic lipids is thought to result from enhanced affinity for cells, many of which bear a net negative charge. Additionally, the net positive charge on lipid aggregates comprising a cationic lipid enables the aggregate to bind polyanions, such as nucleic acids. Lipid aggregates containing DNA are known to be effective agents for efficient transfection of target cells.

Liposomes are microscopic vesicles consisting of concentric lipid bilayers. The lipid bilayers of liposomes are generally organized as closed concentric lamellae, with an aqueous layer separating each lamella from its neighbor. Vesicle size typically falls in a range of between about 20 and about 30,000 nm in diameter. The liquid film between lamellae is usually between about 3 and 10 nm thick.

The structure of various types of lipid aggregates varies, depending on composition and method of forming the aggregate. Such aggregates include liposomes, unilamellar vesicles (ULVs), multilameller vesicles (MLVs), micelles and the like, having particular sizes in the nanometer to micrometer range. Methods of making lipid aggregates are by now well-known in the art. The main drawback to use of conventional phospholipid containing liposomes for delivery is that the material to be delivered must be encapsulated and the liposome composition has a net negative charge which is not attracted to the negatively charged cell surface. By combining cationic lipid compounds with a phospholipid, positively charged vesicles and other types of lipid aggregates can bind DNA, which is negatively charged, and can be taken up by and can transfect target cells. See, for example, Felgner et al., Proc. Natl. Acad. Sci. USA 84, 7413-7417 (1987); U.S. Pat. Nos. 4,897,355 and 5,171,678 and International Publication No. WO 00/27795.

Liposomes may be prepared by a number of methods. Preparing MLV liposomes usually involves dissolving the lipids in an appropriate organic solvent and then removing the solvent under a gas or air stream. This leaves behind a thin film of dry lipid on the surface of the container. An aqueous solution is then introduced into the container with shaking in order to free lipid material from the sides of the container. This process disperses the lipid, causing it to form into lipid aggregates or liposomes. ULV liposomes may be made by slow hydration of a thin layer of lipid with distilled water or an aqueous solution of some sort.

Liposomes may also be prepared by lyophilization. This process comprises drying a solution of lipids to a film under a stream of nitrogen. This film is then dissolved in a volatile solvent, frozen, and placed on a lyophilization apparatus to remove the solvent. To prepare a pharmaceutical formulation containing a drug or other substance, a solution of the substance is added to the lyophilized lipids, whereupon liposomes are formed.

A variety of methods for preparing various liposomes have been described in the periodical and patent literature. For specific reviews and information on liposome formulations, reference is made to reviews by Pagano et al., Ann. Rev. Biophysic. Bioeng., 7, 435-68 (1978) and Szoka et al., Ann. Rev. Biophysic. Bioeng., 9, 467-508 (1980) and to U.S. Pat. Nos. 4,229,360; 4,224,179; 4,241,046; 4,078,052; and 4,235,871.

Various biological substances have been encapsulated into liposomes by contacting a lipid with the matter to be encapsulated and then forming the liposomes as described above. A drawback of these methods is that the fraction of material encapsulated into the liposome structure is generally less than 50%, usually less than 20%, often necessitating an extra step to remove unencapsulated material. An additional problem, related to the above, is that after removal of unencapsulated material, the encapsulated material can leak out of the liposome. This second issue represents a substantial stability problem to which much attention has been addressed in the art.

Despite advances in the field, a need remains for a variety of improved lipid compounds. Since different cell types differ from one another in membrane composition, different compositions and types of lipid aggregates have been found to be effective for different cell types, either for their ability to contact and fuse with target cell membranes, or for aspects of the transfer process itself. At present these processes are not well understood, consequently the design of effective liposomal precursors is largely empirical. Besides content and transfer, other factors are of importance include the ability to form lipid aggregates suited to the intended purpose, the possibility of transfecting cells in the presence of serum, toxicity to the target cell, stability as a carrier for the compound to be delivered, and ability to function in an in vivo environment. In addition, lipid aggregates can be improved by broadening the range of substances which can be delivered into cells.

There still exists a need for improved compositions for delivering genetic material into cells.

### SUMMARY OF THE INVENTION

According to a first aspect of the invention, a composition for transfecting genetic material into cells and a method of transfecting cells with the composition is provided. The composition includes an amphipathic compound and an enzyme inhibitor. The amphipathic compound has a general structure represented by the formula:

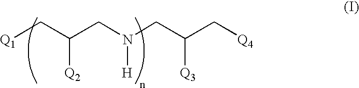

wherein:

n is 0 or a positive integer;

Q₁ is N(R)₃+, N(R)₂, O(R), or O(R)₂+wherein each R substituent is independently selected from the group consisting of H, a straight chain or branched alkyl or alkenyl, a straight chain or branched alkyl or alkenyl ether, a straight chain or branched alkyl or alkenyl ester, a straight chain or branched alkyl or alkenyl carbonyldioxide, a sterol, a lipid, and a hydrophobic hormone with the proviso that at least one R substituent on the O or N atom of Q₁ is not H;

Q₃, and each Q₂ are independently selected from the group consisting of H, O(R′), N(R′)₂, NH(R″), and S(R′); and

Q₄ is selected from the group consisting of N(R′)₂, and NH(R″); wherein:

R′ is H or one the following moieties:

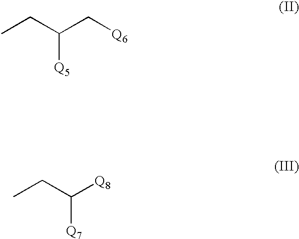

and wherein each of Q₅, Q₆ Q₇ and Q₈ are independently selected from the group consisting of N(R)₃+, N(R)₂, OR, O(R)₂+, O(R′), N(R′)₂, NH(R″), S(R), S(R)₂+ and S(R′); wherein each R substituent on Q₅, Q₆, Q₇ or Q₈ is independently selected from H or a methyl group;

each R′ substituent on Q₅, Q₆, Q₇ or Q₈ is as defined above for Q₄; and

each R″ substituent on Q₂, Q₃ Q₄, Q₅, Q₆, Q₇ or Q₈ is independently hydrogen or comprises a moiety selected from the group consisting of amino acid residues, polypeptide residues, protein residues, carbohydrate residues and combinations thereof. The composition can also include a genetic material such as a DNA plasmid. The genetic material can be an expression vector containing a DNA segment encoding a protein or an anti-sense oligonucleotide. Further, the enzyme inhibitor can be encapsulated in a liposome formed by the amphipathic compound. The enzyme inhibitor is preferably a histone deacetylase inhibitor. The enzyme inhibitor can be trichostatin A (TSA), FR901464, FR901228, and trapoxin A (TPX).

According to a second aspect of the invention, a composition for transfecting genetic material into cells is provided wherein the composition consists essentially of: an amphipathic compound; an enzyme inhibitor; and a pharmaceutically acceptable carrier. According to this aspect of the invention, the enzyme inhibitor can be encapsulated in a liposome formed by the amphipathic compound. A method of transfecting cells with this composition is also provided wherein the method comprises: combining the genetic material with the liposome encapsulated enzyme inhibitor to form a complex between the liposome and the genetic material and incubating one or more cells with the liposome/genetic material complex.

According to a third aspect of the invention, a composition for transfecting genetic material into cells and a method of transfecting cells with the composition is provided wherein the composition comprises a liposome formed by an amphipathic compound; an enzyme inhibitor; and a genetic material. According to this aspect of the invention, the genetic material is not encapsulated in the liposome.

### BRIEF DESCRIPTION OF THE DRAWINGS

The present invention may be better understood with reference to the accompanying drawings in which:

FIG. 1 illustrates a method of forming a complex of genetic material (i.e., DNA), an amphipathic compound (e.g., a cationic lipid) and an inhibitor according to the invention wherein the inhibitor is encapsulated in a liposome;

FIG. 2 illustrates a method of transfecting a cell according to the invention with the complex of FIG. 1;

FIG. 3 is a bar chart showing β-galactosidase activity for Hela cells transfected with a plasmid DNA expression vector using transfection compositions according to the invention;

FIG. 4 is a bar chart showing β-galactosidase activity for COS7 cells transfected with a plasmid DNA expression vector using transfection compositions according to the invention; and

FIG. 5 is a bar chart showing β-galactosidase activity for 293 cells transfected with a plasmid DNA expression vector using transfection compositions according to the invention.

### DETAILED DESCRIPTION OF THE PREFERRED EMBODIMENTS

According to the present invention, compositions that enhance gene transfer into cells and increase protein expression and methods of using these compositions are provided. The compositions according to the invention comprise an amphipathic compound and an enzyme inhibitor (e.g., a histone deacetylase inhibitor). The compositions according to the invention can also comprise a genetic material (e.g., DNA or an oligonucleotide).

The present inventors have found that cultured cells exposed to compositions comprising a genetic material (e.g., DNA or an oligonucleotide), an amphipathic compound, and an enzyme inhibitor (e.g., a histone deacetylase inhibitor) expressed foreign genes at very high levels. The present inventors have also found that the use of histone deacetylase inhibitors and certain amphipathic compounds in combination significantly increased gene transfer efficiency and protein expression.

The formulations according to the invention provide an improved method for transfecting cells and expressing protein at high efficiencies. Further, since the inhibitors used in this invention can be anti-cancer drug candidates, compositions according to the invention can be used in cancer-gene therapy.

The present invention provides a method of transfecting a cell with DNA or other genetic material. The method comprises exposing the cell to a composition comprising the genetic material, an amphipathic compound and an enzyme inhibitor.

Preferred enzyme inhibitors are histone deacetylase inhibitors. Histone deacetylase inhibitors are disclosed in U.S. Pat. No. 5,834,249 as a procedure for the production of protein as well as in Nakajima et al., “FR901228, A potent Antitumor Antibiotic, is a Novel Histone Deacetylase Inhibitor”, Experimental Cell Research, 241, 126-133 (1998) and Yamano et al., “Amplification of Transgene Expression in Vitro and in Vivo Using a Novel Inhibitor of Histone Deacetylas”, Molecular Therapy, 1, 6 (2000).

The enzyme inhibitor according to the invention can be a histone deacetylase inhibitor such as trichostatin A (TSA), FR901464, FR901228, or trapoxin A (TPX). These compounds are merely exemplary, however, and other histone deacetylase inhibitors can be used according to the invention. Since histone deacetylase inhibitors have been used for targeting cancer cells in Minucci et al., “A Histone Deacetylase Inhibitor Potentiates Retinoid Receptor Action in Embryonal Carcinoma Cells”, Proc. Natl. Acad. Sci. USA, 94, 11295-11300 (1997), transfection compositions comprising histone deacetylase inhibitors according to the invention can be used in cancer gene therapy.

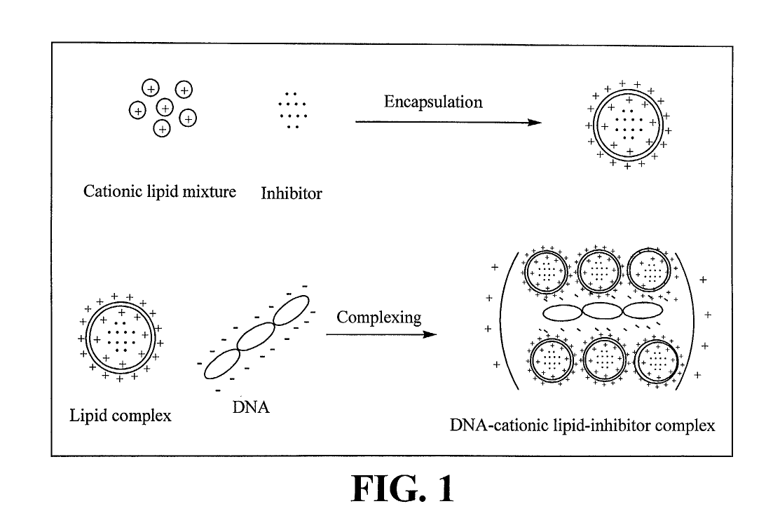

According to a preferred embodiment of the invention, the enzyme inhibitor (e.g., histone deacetylase inhibitor) can be encapsulated into a liposome formed by the amphipathic compound. This procedure is illustrated in FIG. 1. As shown in FIG. 1, an enzyme inhibitor (e.g., histone deacetylase inhibitor) is encapsulated in a liposome formed by a cationic lipid. The resulting liposome is then complexed with a genetic material (e.g., a plasmid DNA) to form the genetic material-lipid-enzyme inhibitor complex.

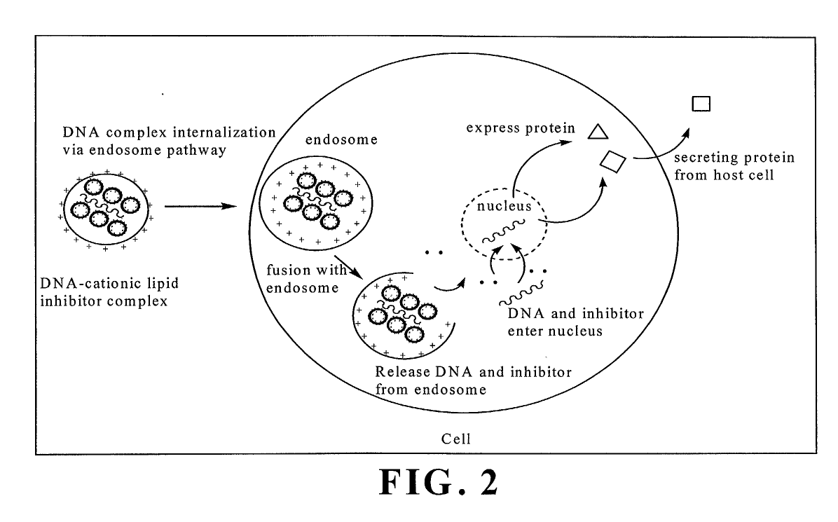

As shown in FIG. 2, the genetic material-lipid-enhancer complex can be internalized into the cytosol of the cell via an endosome pathway. Once in the cytosol, the genetic material and enhancer can be released from the endosome and can enter the nucleus. Once inside the nucleus, the genetic material can express a protein which can, in turn, be secreted from the cell.

Histone deacetylase inhibitors can also be mixed with a pre-formed liposome according to the invention. The resulting composition can then be complexed with a genetic material such as a plasmid DNA.

A variety of amphipathic compounds can be used according to the invention. According to a preferred embodiment of the invention, the amphipathic compound is cationic. Although cationic amphipathic compounds are preferred, non-ionic amphipathic compounds can also be used. Further, mixtures of non-ionic and cationic amphipathic compounds can also be used according to the invention.

The amphipathic compound can be a non-natural (i.e., a synthetic) polyamine wherein one or more of the amines is bonded to at least one hydrophobic moiety. The hydrophobic moiety can be a C6-C24 alkane, a C6-C24 alkene, a sterol, a steroid, a lipid, a fatty acid or a hydrophobic hormone. The amphipathic compounds according to the invention may form liposomes, micelles or clusters.

Several classes of amphipathic compounds are described below. Any of these materials can be used as an amphipathic compound according to the invention.

The following structures are exemplary of amphipathic compounds that can be used according to the invention.

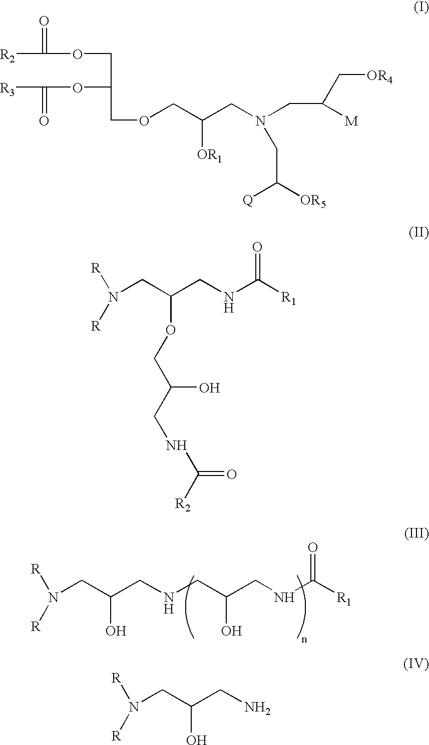

These and other amphipathic compounds suitable for use in the present invention are described in copending U.S. patent application Ser. No. 10/035,223, filed Jan. 4, 2002, which is hereby incorporated by reference in its entirety. Methods for synthesizing these compounds can also be found in U.S. patent application Ser. No. 10/035,223.

According to one embodiment of the invention, a composition for transfecting genetic material into cells is provided wherein the composition consists essentially of: one or more amphipathic compounds; an enzyme inhibitor; and a pharmaceutically acceptable carrier. The phrase “consisting essentially of” in the context of this embodiment of the invention is defined as excluding the presence of genetic material but does not otherwise restrict the contents of the composition. For example, the composition may further comprise a carrier (e.g., a pharmaceutically acceptable carrier) such as water or liposome forming compounds such as DOPE.

The present inventors have found that compositions comprising transfection reagents, enzyme inhibitors (e.g., histone deacetylase inhibitors) and genetic material according to the invention can provide enhanced expression compared to compositions of genetic material and transfection reagent alone.

The chemical structures of exemplary histone deacetylase inhibitors that can be used according to the invention are shown below.

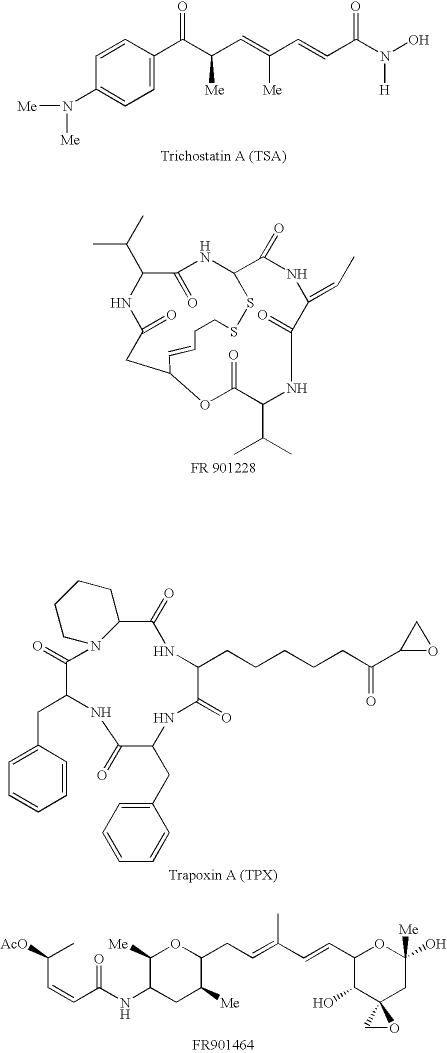

### EXAMPLES

The following examples are intended to further illustrate the invention. Unless otherwise indicated, the lipid compound used in the examples has the following structure:

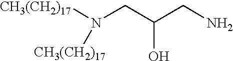

The structure of DOPE, which was used in the examples to form liposomes from the lipid compound, is shown below:

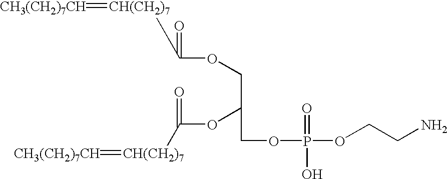

Reagent 1—Inhibitor Entrapped Formulation

A solution of DOPE (30 mg) in 3 ml dichloromethane was mixed with a solution of cationic lipid (45 mg) in 4.5 ml of dichloromethane to form an organic solution. Afterward, 10 ml DCM, 50 ml deionized water and 714 μg of trichostatin A (TSA) in 0.25 ml of DMSO was added to the lipid solution. The two-phase liquids were then mixed vigorously. The organic solvent was removed via rotary-evaporator, and a homogenous liposome was thereby formed. The final volume of the reagent was adjusted to 50 ml. The liposome formulation was then dialyzed against deionized water three times to remove the free TSA and lipids. There was no change in dialysis sample's volume. Also, the lipid formulation remains intact as verified by High Pressure Liquid Chromatography analysis and Thin Layer Chromatography analysis.

### Reagent 2—Inhibitor with Pre-Formulated Lipid Complex

A solution of DOPE (30 mg) in 3 ml dichloromethane was mixed with a solution of cationic lipid (45 mg) in 4.5 ml of dichloromethane to form an organic solution. Afterward, 10 ml DCM and 40 ml of deionized water was added to the lipid solution. The two-phased liquids were mixed vigorously. The organic solvent was then removed via rotary-evaporator, and homogenous liposome was formed. The final volume of the reagent was then adjusted to 40 ml and the liposome formulation was dialyzed against deionized water three times to remove the free lipids.

Trichostatin A of 1 mg was dissolved in 336 μl of DMSO or 1 ml of ethanol. To this solution, was added 9.66 ml or 9.0 ml of deionized water to make Trichostatin A stock solution at 100 μg/ml. Afterward, 7.14 ml of TSA solution was gently added to the pre-formulated lipid complex to get the final formulation.

Reagent 3—Cationic Lipid and DOPE.

A solution of DOPE (30 mg) in 3 ml dichloromethane was mixed with a solution of cationic lipid (45 mg) in 4.5 ml of dichloromethane to form an organic solution. Afterward, 10 ml DCM and 50 ml of deionized water were added to the lipid solution. The two-phased liquids were then mixed vigorously. The organic solvent was removed via rotary-evaporator and a homogenous liposome was formed. The final volume of the reagent was then adjusted to 50 ml and the liposome formulation was dialyzed against deionized water three times to remove the free lipids.

Preparation of Cells

The cell according to the invention can be a mammalian cell that is maintained in tissue culture such as cell lines that are immortalized or transformed. These include a number of cell lines that can be obtained from American Tissue Culture Collection of Bethesda, Md. Suitable cells include 293 cells, COS-7 (monkey kidney) cells, and Hela (human cervical carcinoma) cells.

The mammalian cell can be primary or secondary which means that it has been maintained in culture for a relatively short time after being obtained from an animal tissue.

Both the primary cells and cell lines are grown (cultured) in tissue culture media such as Dulbeco's Modified MEM media (D-MEM, Invitrogen) supplemented with 10% fetal calf serum for COS-7, Hela and 293. The cultures were maintained in a humidified atmosphere of 5% CO₂ in air at 37° C. The cells were then seeded in 24-well plates (culture dishes) 24 h before the transfection at 40-60% confluence.

Preparation of Polynucleotides

The genetic material according to the invention can be a polynucleotide such as a deoxyribonucleic acid (DNA) in the form of an oligonucleotide, anti-sense, plasmid DNA, parts of a plasmid DNA or genetic material derived from a virus. The polynucleotide can also be a ribonucleic acid (RNA).

The exogenous genetic construction is a plasmid DNA that consists of DNA from another organism of the same or different species. The plasmid DNA constructions normally include a coding sequence for a transcription product or a protein of interest, together with flanking regulatory sequences effective to cause the expression of the protein in the transfected cells. Examples of flanking regulatory sequences are a promoter sequence sufficient to initiate transcription and a terminator sequence sufficient to terminate the gene product, by termination of transcription or translation. Suitable transcriptional or translational enhancers can be included in the exogenous gene construct to further assist the efficiency of the overall transfection process and expression of the protein in the transfected cells.

A marker or reporter gene encodes a gene product that can be easily assayed, such as β-galactosidase. The presence of the product of the marker gene indicates that the cell is transfected and the amount of the product indicates the transfection efficiency.

Plasmid pCMV.SPORT-βgal contains the β-galactosidase (β-gal) gene from E. coli cloned as a Not I fragment into plasmid pCMV.SPORT 1. The plasmid contains the CMV promoter. An SV40 polyadenylation signal downstream of the β-gal gene directs proper processing of the mRNA in eukaryotic cells.

Example of Gene Transfection Experiments:

The complete media used for cells in the gene transfection experiments described below was DMEM with 10% FBS. The β-gal plasmid is pCMV-SPORT-β-gal plasmid. The cells were plated in 24-well plates at a density of 1×10⁴ cells/well in 1 mL of complete media per well and place plates in a 37° C., 5% CO₂ humidified incubator. After 24 hours, 1 μg of DNA was diluted in sterile deionized water or serum free medium to a total volume of 10 μl. The solution was then mixed and spun down for a few seconds to remove drops from the top of the tube. Afterward, 4 μl of the cationic lipid transfection reagent was added to the DNA solution containing 7 μl of sterile deionized water or serum free medium. The contents of the tube were then mixed by pipetting up and down 6 times. The solutions were then allowed to incubate for 10 minutes at 20-25° C. to allow transfection-DNA complex formation. Afterward, 20 μl transfection-DNA complex was mixed with 180 μl Opti-MEM and then immediately add to the appropriate well by drop-wise fashion. The dish was then gently swirled to ensure uniform distribution of the transfection complexes and the cells were put back into the incubator. For transient transfections, cells were assayed for expression of the transfected gene 24 to 48 hrs after transfection. For stable transfections, cells were passaged 1:4 to 1:8 into the appropriate selective medium 24-48 hours after transfection.

The inhibitor (e.g., TSA) solution may also be added into cell culture medium 20 minutes before adding the transfection-DNA complex.

Hela, COS-7 and 293 cells were transfected with compositions comprising enzyme inhibitor (e.g., TSA), DNA plasmid, and lipid (Reagents 1 and 2) as well as with compositions comprising only the lipid and DNA plasmid (Reagent 3) and compositions comprising only the inhibitor (e.g., TSA) and DNA plasmid (TSA+DNA). The results are shown below in Tables 1, 2 and 3.

TABLE 1
The transfection efficiency of different formulations in Hela Cells

| | β-Gal Activity Assay |
|---|---|
| Complex Name | (ng β gal/cm²) |
| Reagent 1 | 478 |
| Reagent 2 | 601 |
| Reagent 3 | 397 |
| TSA + DNA | 5 |

TABLE 2
The transfection efficiency of different
formulations in COS-7 Cells

| | β-Gal Activity Assay |
|---|---|
| Complex Name | (ng β gal/cm²) |
| Reagent 1 | 267 |
| Reagent 2 | 63 |
| Reagent 3 | 46 |
| TSA + DNA | 0.45 |

TABLE 3
The transfection efficiency of different
formulations in 293 Cells

| | β-Gal Activity Assay |
|---|---|
| Complex Name | (ng β gal/cm²) |
| Reagent 1 | 655 |
| Reagent 2 | 193 |
| Reagent 3 | 73 |
| TSA + DNA | 0.5 |

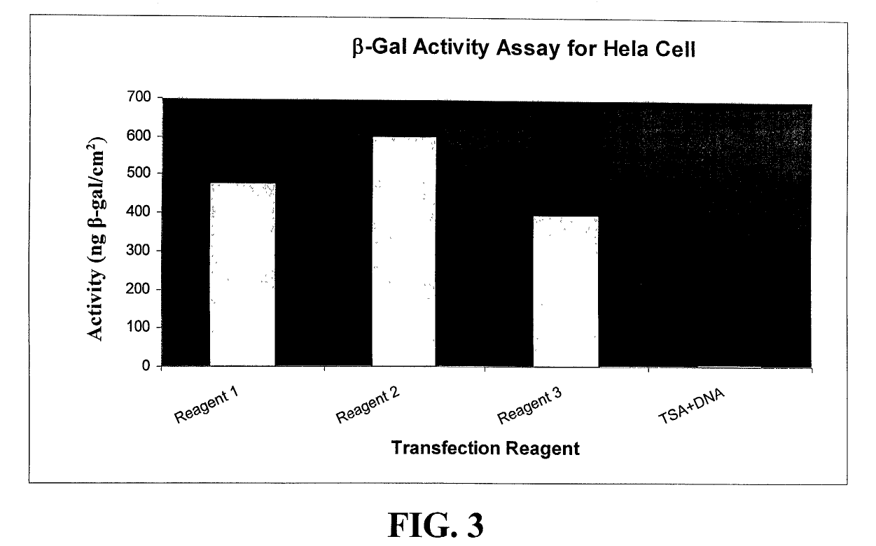

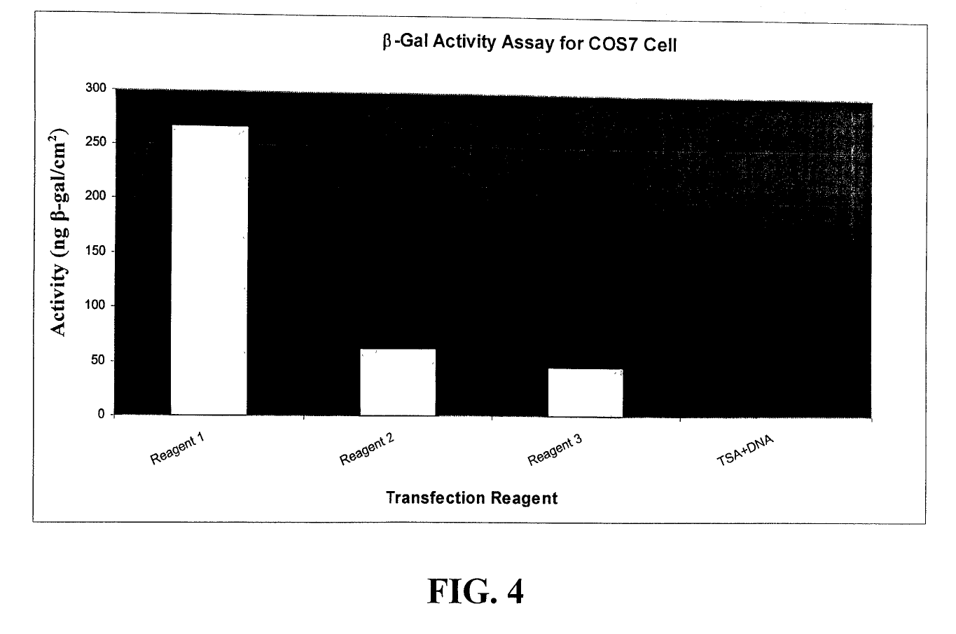

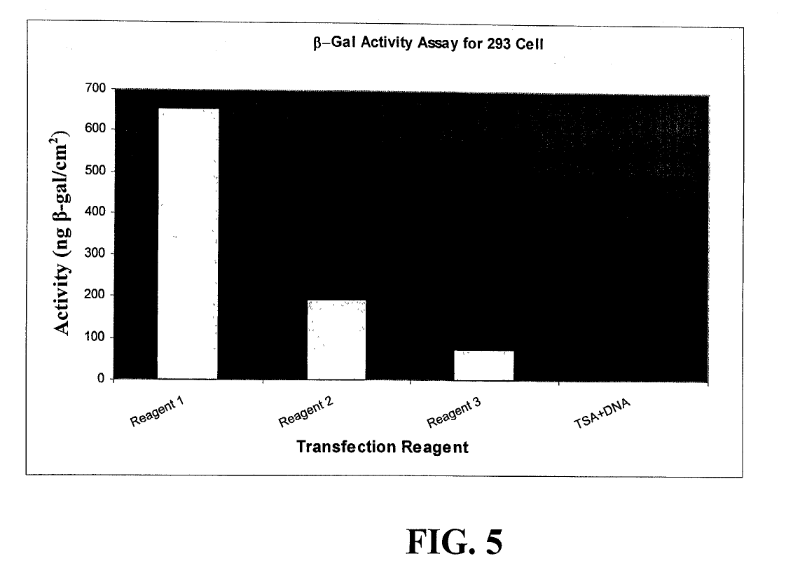

The data from Tables 1, 2 and 3 are shown in bar chart form in FIGS. 3, 4 and 5 respectively. As can be seen from FIGS. 3, 4, and 5, the gene expression level increases dramatically in Hela, COS7 and 293 cells, respectively, when a composition according to the invention comprising an enzyme inhibitor, a genetic material and an amphipathic compound (e.g., a lipid) is used to transfect the genetic material into the cell. In particular, the reporter gene was expressed at much higher levels when a composition comprising a lipid and an enzyme inhibitor in addition to the DNA plasmid was used. In fact, as shown in FIGS. 3, 4 and 5, there is no observable gene expression when only the inhibitor (e.g., TSA) is used with the DNA plasmid in 293, COS-7, and Hela cell lines. Further, the use of a composition comprising a liposome encapsulated inhibitor (Reagent 1) resulted in much higher levels of gene expression in COS7 cells than a composition comprising a non-encapsulated inhibitor (Reagent 2). In all cell types, the presence of enzyme inhibitor in the composition increased gene expression.

From the above data, it appears that TSA functions only as a transcription enhancer and not as a transfection agent. As shown in FIGS. 3, 4 and 5, TSA cannot transfect DNA plasmid without the presence of the lipid transfection reagent. The presence of an enzyme inhibitor such as TSA can, however, significantly increase protein expression when used in combination with a lipid transfection reagent according to the invention.

Compounds that can be transfected using compositions according to the invention include DNA, RNA, oligonucleotides, peptides, proteins, carbohydrates and drugs. Methods of transfection and delivery of these and other compounds are well-known in the art.

As set forth above, amphipathic compounds according to the invention can be formed into aggregates (e.g., liposomes). Various techniques for forming liposomes are known in the art. See, for example, Zadi et al., “A Novel Method for High-Yield Entrapment of Solutes Into Small Liposomes”, Liposome Research, 10, 73-80 (2000). Lipid aggregates according to the invention can be formed using a lipid aggregate forming compound such as DOPE, DOPC or cholesterol.

Other substances such as proteins, peptides and growth factors can also be added to the compositions according to the invention to enhance cell targeting, uptake, internalization, nuclear targeting and expression.

Compositions according to the invention may also be provided in a kit comprising the amphipathic compound, the enzyme inhibitor and at least one additional component. The additional component can be one or more cells, a cell culture media, a genetic material (e.g., a nucleic acid) or a transfection enhancer.

According to a preferred embodiment of the invention, the transfection enhancer can be a biodegradable polymer such as a natural polymer, a modified natural polymer, or a synthetic polymer. Suitable biodegradable polymers include, but are not limited to, carbohydrates (e.g., linear or T-shaped carbohydrates) and polysaccharides such as amylopectin, hemi-cellulose, hyaluronic acid, amylose, dextran, chitin, cellulose, heparin and keratan sulfate. The transfection enhancer according to the invention can also be a DNA condensing protein (e.g., a histone or a protamine), a cell membrane disruption peptide or a ligand (e.g., a peptide or a carbohydrate) which specifically targets certain surface receptors on the cell being transfected. For example, the ligand can interact with surface receptors on the cell being transfected via ligand and receptor interactions. In this manner, transfection can be enhanced (e.g., via receptor mediated endocytosis).

The compositions of the present invention can yield lipid aggregates that can be used in the same manner as other known transfection agents. For example, a liposome can be formed from lipid compounds according to the invention and the liposome can be contacted with a substance to be transfected to form a complex between the liposome and the substance. The complex can then be incubated with one or more cells.

The transfection methods according to the invention can be applied to in vitro or in vivo transfection of cells, particularly to the transfection of eukaryotic cells or tissue including animal cells, human cells, insect cells, plant cells, avian cells, fish cells, mammalian cells and the like.

The methods of the invention can also be used to generate transfected cells or tissues which express useful gene products. For example, the methods of the invention can be used to produce transgenic animals. The methods of the invention are also useful in any therapeutic method requiring the introduction of nucleic acids into cells or tissues, particularly for cancer treatment, in vivo and ex vivo gene therapy and in diagnostic methods. Methods of this type are disclosed, for example, in U.S. Pat. No. 5,589,466 which is herein incorporated by reference in its entirety.

The compounds and methods of the invention can also be employed in any transfection of cells done for research purposes. Nucleic acids that can be transfected by the methods of the invention include DNA and RNA from any source including those encoding and capable of expressing therapeutic or otherwise useful proteins in cells or tissues, those which inhibit expression of nucleic acids in cells or tissues, those which inhibit enzymatic activity or which activate enzymes, those which catalyze reactions (ribozymes) and those which function in diagnostic assays.

The compositions and methods of the invention can also be readily adapted to introduce biologically active macromolecules or substances other than nucleic acids into cells. Suitable substances include polyamines, polyamino acids, polypeptides, proteins, biotin and polysaccharides. Other useful materials such as therapeutic agents, diagnostic materials and research reagents can also be introduced into cells by the methods of the invention.

It will be readily apparent to those of ordinary skill in the art that a number of general parameters can influence the efficiency of transfection or delivery. These parameters include, for example, the lipid concentration, the enzyme inhibitor concentration, the concentration genetic material to be delivered, the number of cells transfected, the medium employed for delivery, the length of time the cells are incubated with the composition, and the relative amounts of cationic and non-cationic lipid. It may be necessary to optimize these parameters for each particular cell type. Such optimization can be routinely conducted by one of ordinary skill in the art employing the guidance provided herein and knowledge generally available to the art.

It will also be apparent to those of ordinary skill in the art that alternative methods, reagents, procedures and techniques other than those specifically detailed herein can be employed or readily adapted to produce the liposomal precursors and transfection compositions of this invention. Such alternative methods, reagents, procedures and techniques are within the spirit and scope of this invention.

From the foregoing, it will be appreciated that, although specific embodiments of the invention have been described herein for purposes of illustration, various modifications may be made without deviating from the spirit and scope of the invention.

What is claimed is:

1. A composition for transfecting cells comprising:
an amphipathic compound; and
an enzyme inhibitor;
wherein the amphipathic compound has a general structure represented by the formula: 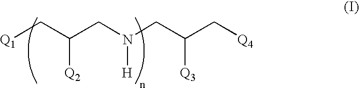
wherein:
n is 0 or a positive integer;
Q₁ is N(R)₃+, N(R)₂, O(R), or O(R)₂+ wherein each R substituent is independently selected from the group consisting of H, a straight chain or branched alkyl or alkenyl, a straight chain or branched alkyl or alkenyl ether, a straight chain or branched alkyl or alkenyl ester, a straight chain or branched alkyl or alkenyl carbonyldioxide, a sterol, a lipid, and a hydrophobic hormone with the proviso that at least one R substituent on the O or N atom of Q₁ is not H;
Q₃, and each Q₂ are independently selected from the group consisting of H, O(R′), N(R′)₂, NH(R″), and S(R′); and
Q₄ is selected from the group consisting of N(R′)₂, and NH(R″); wherein:
R′ is H or one the following moieties: 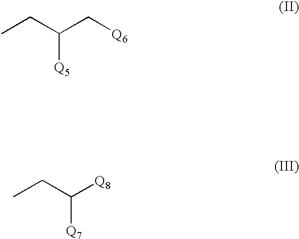
and wherein each of Q₅, Q₆, Q₇ and Q₈ are independently selected from the group consisting of N(R)₃+, N(R)₂, OR, O(R)₂+, O(R′), N(R′)₂, NH(R″), S(R), S(R)₂+ and S(R′); wherein each R substituent on Q₅, Q₆, Q₇ or Q₈ is independently selected from H or a methyl group;
each R′ substituent on Q₅, Q₆, Q₇ or Q₈ is as defined above for Q₄; and
each R″ substituent on Q₂, Q₃, Q₄, Q₅, Q₆ Q₇ or Q₈ is independently hydrogen or comprises a moiety selected from the group consisting of amino acid residues, polypeptide residues, protein residues, carbohydrate residues and combinations thereof.

2. The composition of claim 1, further comprising a genetic material.

3. The composition of claim 2, wherein the genetic material is a DNA plasmid.

4. The composition of claim 1, wherein the amphipathic compound is cationic.

5. The composition of claim 1, wherein the enzyme inhibitor is encapsulated in a liposome formed by the amphipathic compound.

6. The composition of claim 5, further comprising a genetic material complexed to the liposome.

7. The composition of claim 6, wherein the genetic material is a DNA plasmid.

8. The composition of claim 2, wherein the genetic material comprises an expression vector comprising a DNA segment encoding a protein or an anti-sense oligonucleotide.

9. The composition of claim 1, wherein the enzyme inhibitor is selected from the group of histone diacetylase inhibitors consisting of trichostatin A (TSA), FR901464, FR901228, and trapoxin A (TPX).

10. The composition of claim 1, wherein the amphipathic compound has the following structure: 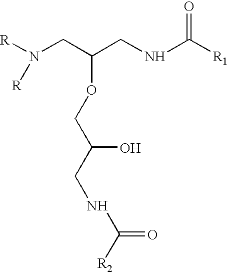
wherein:
each R is a hydrophobic moiety independently selected from the group consisting of a C6-C24 alkane, a C6-C24 alkene, a sterol, a steroid, a lipid, a fatty acid, and a hydrophobic hormone;
R₁ and R₂ are cationic groups independently selected from the group consisting of polyamines, cationic peptides, cationic DNA binding proteins, NLS conjugated cationic peptides and NLS conjugated cationic DNA binding proteins.

11. The composition of claim 10, wherein R₁ and R₂ are independently histones or protamines.

12. The composition of claim 1, wherein the amphipathic compound has the structure: 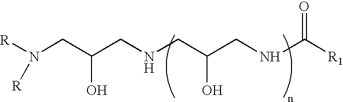
wherein:
n=0, or a positive integer;
each R is a hydrophobic moiety independently selected from the group consisting of a C6-C24 alkane, a C6-C24 alkene, a sterol, a lipid, and a hydrophobic hormone; and
R₁ is a cationic group selected from the group consisting of polyamines, cationic peptides, cationic DNA binding proteins, NLS conjugated cationic peptides and NLS conjugated cationic DNA binding proteins.

13. The composition of claim 12, wherein R₁ is a histone or a protamine.

14. The composition of claim 1, wherein the amphipathic compound has the structure: 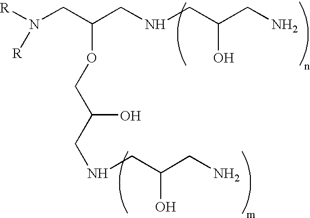
wherein:
each R is a hydrophobic moiety independently selected from the group consisting of a C6-C24 alkane, a C6-C24 alkene, a sterol, a lipid, and a hydrophobic hormone; and
“m” and “n” are 0 or positive integers.

15. The composition of claim 1, wherein the amphipathic compound has the structure: 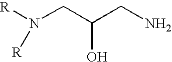
wherein each R is a hydrophobic moiety independently selected from the group consisting of a C6-C24 alkane, a C6-C24 alkene, a sterol, a lipid, and a hydrophobic hormone.

16. The composition of claim 1, wherein the amphipathic compound has the structure: 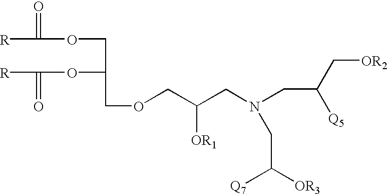
wherein:
each R is a hydrophobic moiety independently selected from the group consisting of a C6-C24 alkane, a C6-C24 alkene, a sterol, a lipid, and a hydrophobic hormone; and
R₁, R₂ and R₃ are independently hydrogen, an alkyl group, or a carbohydrate residue.

17. The composition of claim 1, wherein the amphipathic compound has the structure: 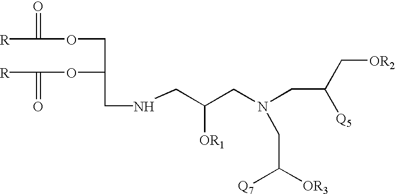
wherein:
each R is a hydrophobic moiety independently selected from the group consisting of a C6-C24 alkane, a C6-C24 alkene, a sterol, a lipid, and a hydrophobic hormone; and
R₁, R₂ and R₃ are independently H, an alkyl group, or a carbohydrate residue.

18. A kit comprising a composition as set forth in claim 1 and at least one additional component selected from the group consisting of one or more cells, a cell culture media, a nucleic acid, a transfection enhancer and combinations thereof.

19. The kit of claim 18, wherein the kit comprises a cell comprising one or more enzymes involved in DNA expression and wherein the enzyme inhibitor inhibits at least one of the one or more enzymes involved in DNA expression.

20. A method for introducing a genetic material into cells, the method comprising incubating one or more cells with a composition comprising:
an amphipathic compound;
an enzyme inhibitor; and
the genetic material;
wherein the amphipathic compound has a general structure represented by the formula: 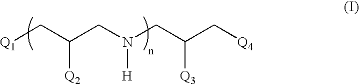
wherein:
n is 0 or a positive integer;
Q₁ is N(R)₃+, N(R)₂, O(R), or O(R)₂+ wherein each R substituent is independently selected from the group consisting of H, a straight chain or branched alkyl or alkenyl, a straight chain or branched alkyl or alkenyl ether, a straight chain or branched alkyl or alkenyl ester, a straight chain or branched alkyl or alkenyl carbonyldioxide, a sterol, a lipid, and a hydrophobic hormone with the proviso that at least one R substituent on the O or N atom of Q₁ is not H;
Q₃, and each Q₂ are independently selected from the group consisting of H, O(R′), N(R′)₂, NH(R″), and S(R″); and
Q₄ is selected from the group consisting of N(R′)₂, and NH(R″); wherein:
R′ is H or one the following moieties: 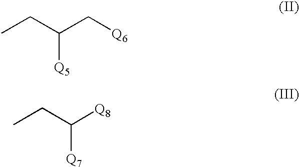
and wherein each of Q₅, Q₆, Q₇ and Q₈ are independently selected from the group consisting of N(R)₃+, N(R)₂,OR, O(R)₂+1O(R′), N(R′)₂, NH(R″), S(R), S(R)₂+ and S(R′); wherein each R substituent on Q₅, Q₆, Q₇ or Q₈ is independently selected from H or a methyl group;
each R′ substituent on Q₅, Q₆, Q₇ or Q₈ is as defined above for Q₄; and
each R″ substituent on Q₂, Q₃, Q₄ Q₅, Q₆, Q₇ or Q₈ is independently hydrogen or comprises a moiety selected from the group consisting of amino acid residues, polypeptide residues, protein residues, carbohydrate residues and combinations thereof.

21. The method of claim 20, wherein the genetic material selected from the group consisting of DNA, RNA, oligonucleotides, DNA plasmids and nucleic acids.

22. The method of claim 20, wherein the enzyme inhibitor is encapsulated in a liposome formed by the amphipathic compound or the enzyme inhibitor can be mixed with pre-formed liposome of the amphipathic compound.

23. The method of claim 20, wherein the genetic material is introduced into the cells in vivo.

24. The method of claim 23, wherein the genetic material is a gene therapy agent and the method is a method of performing gene therapy.

25. The method of claim 24, wherein the genetic material comprises an expression vector comprising a DNA segment encoding a protein or an anti-sense oligonucleotide.

26. The method of claim 24, wherein the gene therapy agent is a cancer gene therapy agent.

27. The method of claim 20, wherein the enzyme inhibitor is selected from the group of histone diacetylase inhibitors consisting of trichostatin A (TSA), FR901464, FR901228, and trapoxin A (TPX).

28. The method of claim 20, wherein the amphipathic compound has the following structure: 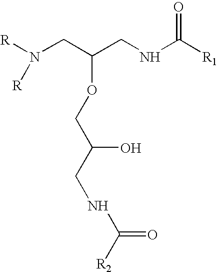
wherein:
each R is independently a hydrophobic moiety selected from the group consisting of a C6-C24 alkane, a C6-C24 alkene, a sterol, a steroid, a lipid, a fatty acid, and a hydrophobic hormone;
R₁ and R₂ are cationic groups independently selected from the group consisting of polyamines, cationic peptides, cationic DNA binding proteins, NLS conjugated cationic peptides and NLS conjugated cationic DNA binding proteins.

29. The method of claim 28, wherein R₁ and R₂ are independently histones or protamines.

30. The method of claim 20, wherein the amphipathic compound has the structure: 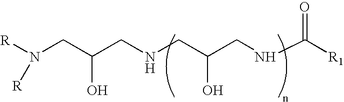
wherein:
each R is independently a hydrophobic moiety selected from the group consisting of a C6-C24 alkane, a C6-C24 alkene, a sterol, a lipid, and a hydrophobic hormone; and
R₁ is a cationic group selected from the group consisting of polyamines, cationic peptides, cationic DNA binding proteins, NLS conjugated cationic peptides and NLS conjugated cationic DNA binding proteins.

31. The method of claim 30, wherein R₁ is a histone or a protamine.

32. The method of claim 20, wherein the amphipathic compound has the structure: 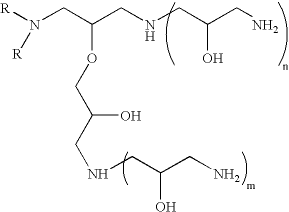
wherein:
each R is a hydrophobic moiety independently selected from the group consisting of a C6-C24 alkane, a C6-C24 alkene, a sterol, a lipid, and a hydrophobic hormone; and
“m” and “n” are 0 or positive integers.

33. The method of claim 20, wherein the amphipathic compound has the structure: 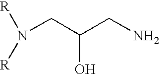
wherein each R is a hydrophobic moiety independently selected from the group consisting of a C6-C24 alkane, a C6-C24 alkene, a sterol, a lipid, and a hydrophobic hormone.

34. The method of claim 20, wherein the amphipathic compound has the structure: 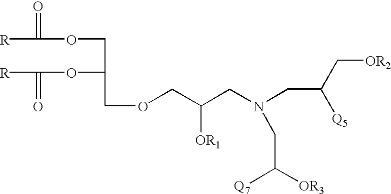
wherein:
each R is a hydrophobic moiety independently selected from the group consisting of a C6-C24 alkane, a C6-C24 alkene, a sterol, a lipid, and a hydrophobic hormone; and
R₁, R₂ and R₃ are independently hydrogen, an alkyl group, or a carbohydrate residue.

35. The method of claim 20, wherein the amphipathic compound has the structure: 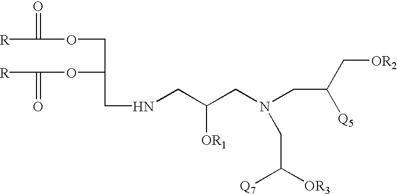
wherein:
each R is a hydrophobic moiety independently selected from the group consisting of a C6-C24 alkane, a C6-C24 alkene, a sterol, a lipid, and a hydrophobic hormone; and
R₁, R₂ and R₃ are independently H, an alkyl group, or a carbohydrate residue.

36. A composition for transfecting cells consisting essentially of:
one or more amphipathic compounds; and
an enzyme inhibitor.

37. The composition of claim 36, wherein the enzyme inhibitor is a histone deacetylase inhibitor.

38. The composition of claim 36, wherein the amphipathic compound is a cationic compound.

39. The composition of claim 36, wherein the amphipathic compound forms a liposome and wherein the enzyme inhibitor is encapsulated in the liposome.

40. A method for introducing a genetic material into cells, the method comprising:
combining the genetic material with the composition of claim 39 to form a complex between the liposome and the genetic material;
incubating one or more cells with the liposome/genetic material complex.

41. A composition for transfecting cells comprising:
a liposome formed by an amphipathic compound;
an enzyme inhibitor;
and a genetic material;
wherein the genetic material is not encapsulated in the liposome.

42. The composition of claim 41, wherein the enzyme inhibitor is encapsulated by the liposome.

43. The composition of claim 41, wherein the amphipathic compound is a cationic compound.

44. The composition of claim 41, wherein the enzyme inhibitor is a histone deacetylase inhibitor selected from the group consisting of trichostatin A (TSA), FR901464, FR901228, and trapoxin A (TPX).

45. A method for introducing a genetic material into cells, the method comprising incubating one or more cells with a composition as set forth in claim 41.

46. The method of claim 45, wherein the enzyme inhibitor is encapsulated in a liposome formed by the amphipathic compound.

47. The method of claim 45, wherein the genetic material is introduced into the cells in vivo.

48. The method of claim 47, wherein the genetic material is a gene therapy agent and the method is a method of performing gene therapy.

49. The method of claim 48, wherein the genetic material comprises an expression vector comprising a DNA segment encoding a protein or an anti-sense oligonucleotide.
